(19) United States
(12) Patent Application Publication
(10) Pub. No.: US 2011/0251455 A1
(43) Pub. Date: Oct. 13, 2011

(54) NESTED CANNULA CONFIGURATION FOR USE WITH ENDOSCOPE

(75) Inventor: Aleksandra Popovic, New York, NY (US)

(73) Assignee: KONINKLIJKE PHILIPS ELECTRONICS N.V., EINDHOVEN (NL)

(21) Appl. No.: 13/139,794

(22) PCT Filed: Nov. 10, 2009
(86) PCT No.: PCT/IB09/54991

§ 371 Date: Jun. 15, 2011

Related U.S. Application Data
(60) Provisional application No. 61/140,174, filed on Dec. 23, 2008.

(51) Int. Cl. A61B 1/00

(52) U.S. Cl. 600/104

(57) ABSTRACT

A method for accessing a target location (61) relative to an anatomical region (41) involves a navigation of a distal end (11) of an endoscope (10) to a cannula insertion location (60) that defines a position and an orientation of the distal end (11) of the endoscope (10) relative to the anatomical region (41). The method further involves an insertion of a nested cannula (20) through an instrument channel (12) of the endoscope (10) to the target location (61) with the nested cannula (20) including a plurality of telescoping tubes (21-24) configured to reach the target location (61) relative to the cannula insertion location (60).

The present invention generally relates to nested cannula configurations that are customized for a patient to facilitate minimally invasive surgical procedures. The present invention specifically relates to a method and a device for accessing small, deep-seeded lesions within the body by planning a 3D path of a nested cannula through an instrument channel of an endoscope.

Endoscopes are widely utilized medical devices. They are used for looking inside the body and are often inserted in body's natural orifices. Besides being used for visual inspection, they often serve as a guide for inserting other devices, such as, for example, catheters or gripping instruments. Therefore, commercial endoscopes usually have one or more “instrument channels” providing insertion path for the instruments.

For purposes of the present invention, the term “endoscope” is broadly defined herein as any device having the ability to image from inside a body. Examples of an “endoscope” include, but are not limited to, a bronchoscope, an arthroscope, a choledochoscope, a colonoscope, a cystoscope, a duodenoscope, a gastroscope, a hysteroscope, a laparoscope, a laryngoscope, a neuroscope, an otoscope, a push enteroscope, a rhinolaryngoscope, a sigmoidoscope, a sinuscope and a thorascope.

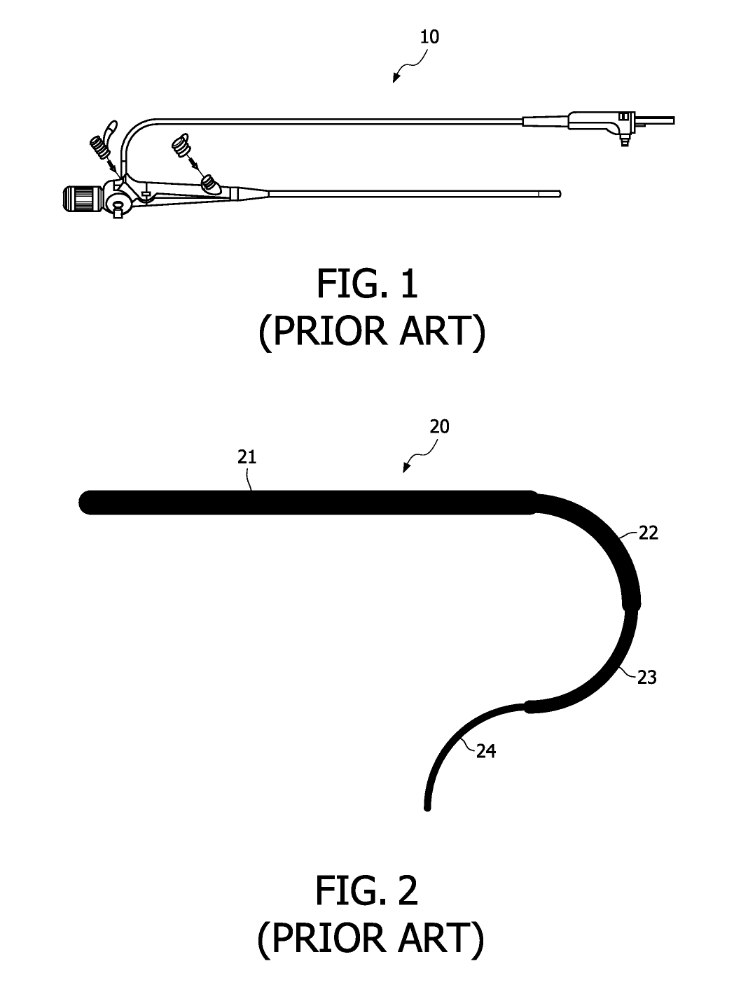

In particular, bronchoscopy is an intra-operative procedure typically performed with a standard bronchoscope (e.g., a bronchoscope 10 shown in FIG. 1) in which the bronchoscope is placed inside of a patient's bronchial tree to provide visual information of the inner structure. Known methods for spatial localization of the bronchoscope include (1) electromagnetic (“EM”) tracking involving an external electromagnetic field generator and the bronchoscope being equipped with sensor coils, (2) optical localization tracking of the bronchoscope involving the bronchoscope being equipped with infrared (“IR”) reflecting spheres, and (3) image based tracking involving a registration of a pre-operative three-dimensional (“3D”) dataset with two-dimensional (“2D”) endoscopic images from a bronchoscope.

The main disadvantages of currently available endoscopes are their size and limited steerability, making them not capable if reaching distant locations within the body. Tracked endoscopes might overcome some of the steerability problems, since they offer on-line position feedback. However, transformation of pre-operative planning to endoscope movement relies on hand-eye coordination of the operator. Tracked endoscopes have the same size as standard endoscopes, making navigation in very small spaces impossible.

International Publication no. WO 2008/032230 A1, Mar. 20, 2008 entitled “Active Cannula Configuration for Minimally Invasive Surgery” to Karen I. Trovato teaches systems and methods related to nested cannula configurations that are customized for a patient to facilitate minimally invasive surgical procedures. Generally, the nested cannula configuration is designed for a specific patient based on a pre-acquired 3D image of a particular anatomical region of the patient, and an identification of a target location within the anatomical region.

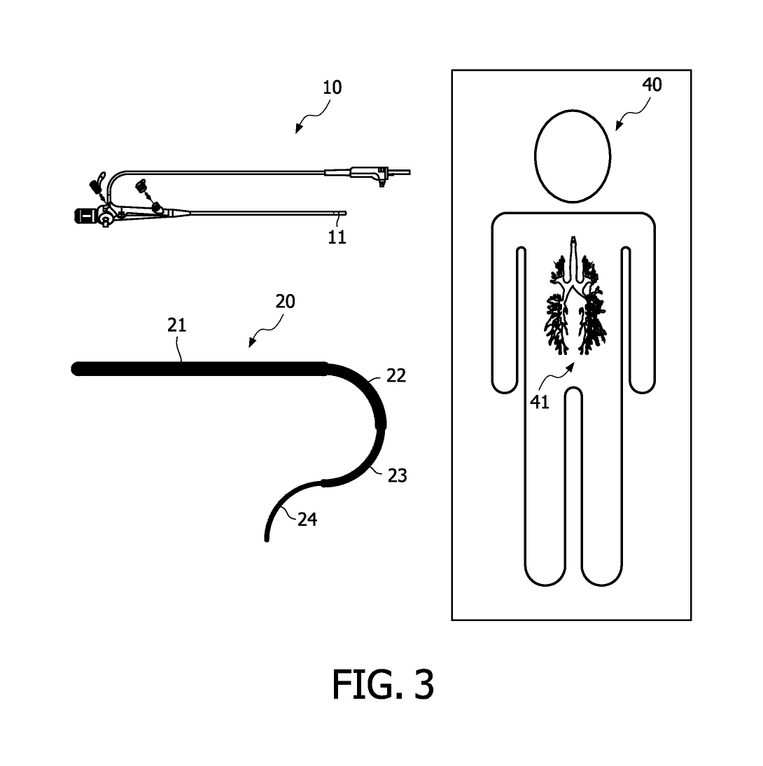

Specifically, nested cannula (“NC”) configurations are designed by utilizing the 3D image to generate a series of arc and straight shapes from a particular position and orientation in the 3D image of the anatomical region. The generated arc and straight shapes are utilized to calculate a pathway between an entry location and the target location. The generated pathway is utilized to generate a plurality of nested telescoping tubes that are configured and dimensioned with pre-set curved shapes. The tubes are typically extended largest to smallest, and the planner specification defines the lengths and the relative orientations between successive tubes to reach the target location. FIG. 2 illustrates an exemplary nested cannula 20 having a straight tube 21 being the largest tube and smaller arc tubes 22-24.

The tubes are fabricated from a material exhibiting desirable levels of flexibility/elasticity. For example, the material may be Nitinol, which has superelastic properties that allow the Nitinol to bend when a force is applied and to return to its original shape once the force is removed.

NC configuration solves both size and steerability problems, allowing pre-operative planning and intra-operative steering of miniaturized hollow tubes (as small as 0.2 mm). The main disadvantage of the NC configuration is that it requires a positioning device to determine the point and the angle of insertion.

The present invention solves both problem of steering/pre-operative planning of instrument movement and pre-positioning of NC configuration.

The present invention is premised on the use of an instrument channel of an endoscope to advance nested cannula tubes towards hard-to-reach lesions within the body. The endoscope is used to explore conventionally reachable areas, whereas the nested cannula performs the final expansion to arrive at the desired target location.

One form of the present invention is a method for accessing a target location relative to an anatomical region involves a navigation of a distal end of an endoscope to a cannula insertion location that defines a position and an orientation of the distal end of the endoscope relative to the anatomical region. The method further involves an insertion of a nested cannula through an instrument channel of the endoscope to the target location with the nested cannula including a plurality of telescoping tubes configured to reach the target location relative to the cannula insertion location.

Another form of the present invention is a device for accessing a target location relative to an anatomical region. The device includes an endoscope having a distal end structurally configured to be navigated to a cannula insertion location that defines a position and an orientation of the distal end of the endoscope relative to the anatomical region. The device further includes a nested cannula operable to be inserted through an instrument channel of the endoscope to the target location with the nested cannula including a plurality of telescoping tubes structurally configured to reach the target location relative to the cannula insertion location.

Another form of the present invention is a system for accessing a target location relative to an anatomical region. The system the aforementioned device and an imaging unit for displaying intra-operative images of the anatomical region including the endoscope and the nested cannula.

The term “pre-operative” as used herein is broadly defined to describe any activity occurring or related to a period or preparations before an endoscopic application (e.g., path planning for an endoscope and configuring a nested cannula), and the term “intra-operative” as used herein is broadly defined to describe any activity occurring, carried out, or encountered in the course of an endoscopic application (e.g., navigating the endoscope derived from the planned path and extending a nested cannula derived from its configuration). Examples of an endoscopic application include, but are not limited to, a bronchoscopy, a colonscopy, a laparascopy, and a brain endoscopy.

The foregoing forms and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

FIG. 1. illustrates a bronchoscope as known in the art.

FIG. 2 illustrates a nested cannula as known in the art.

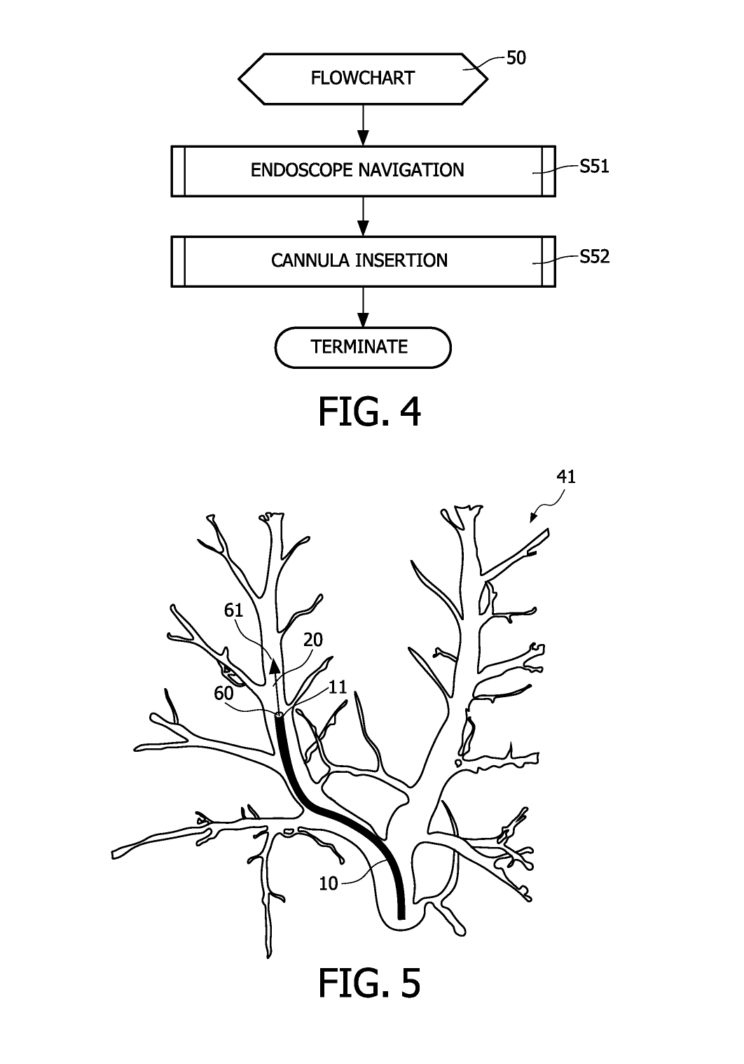

FIG. 3 illustrates an exemplary embodiment of a minimally invasive surgery derived from the present invention.

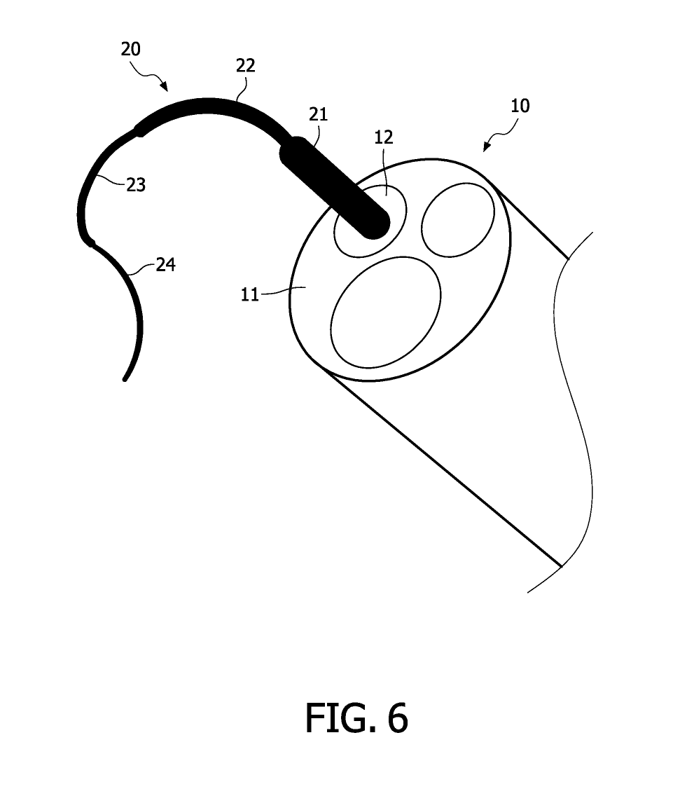

FIG. 4 illustrates a flowchart representative of a minimally invasive surgical method derived from the present invention.

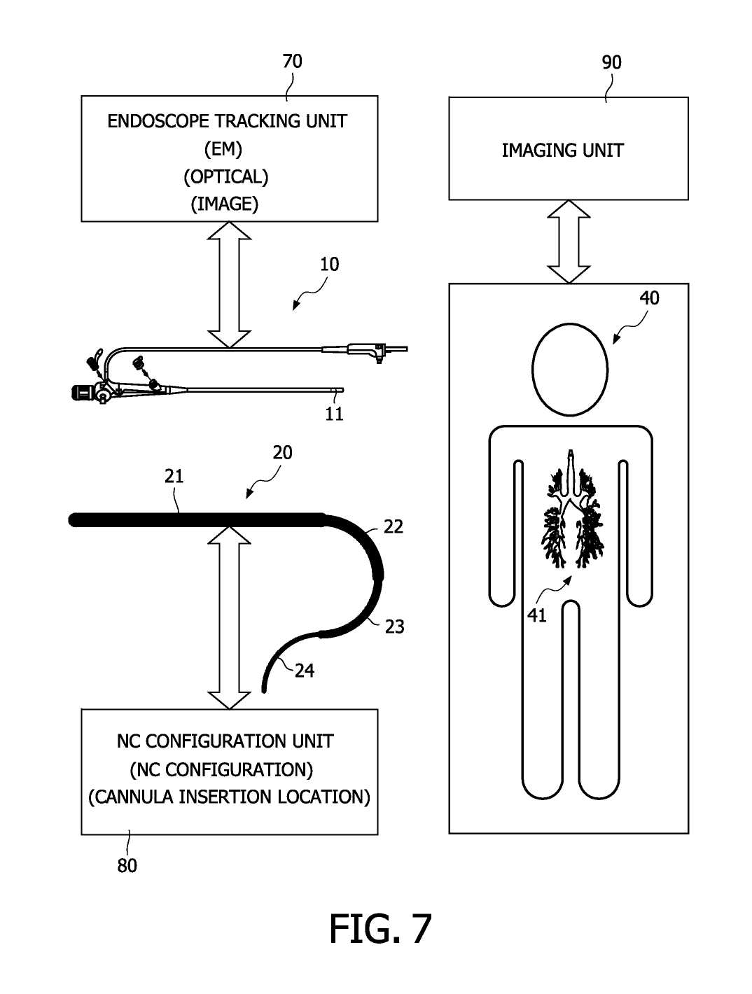

FIG. 5 illustrates an exemplary endoscope navigation and cannula insertion of the minimally invasive surgical method illustrated in FIG. 4.

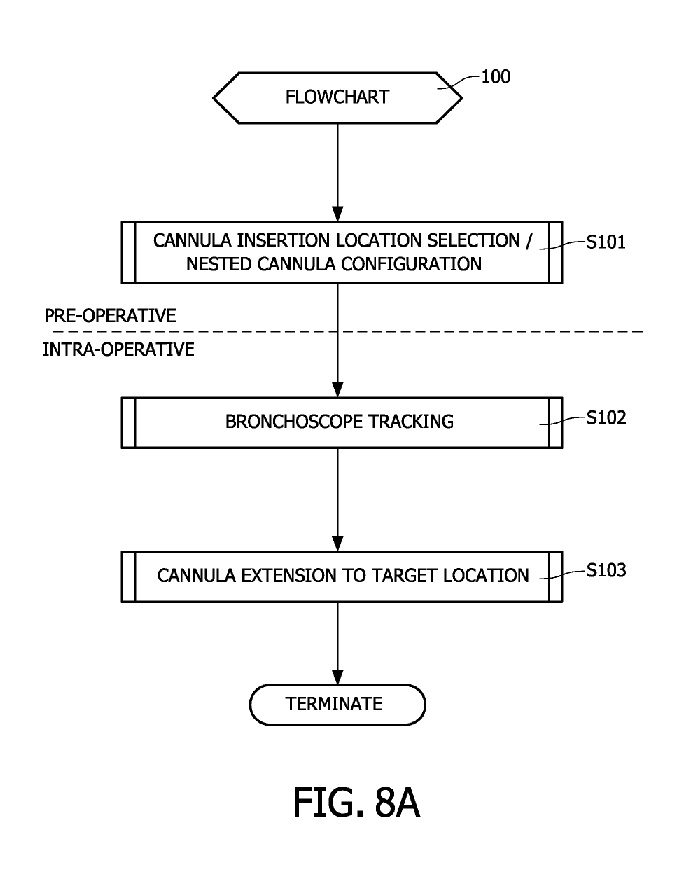

FIG. 6 illustrates an exemplary extension of a nested cannula configuration from a distal end of an endoscope derived from the present invention.

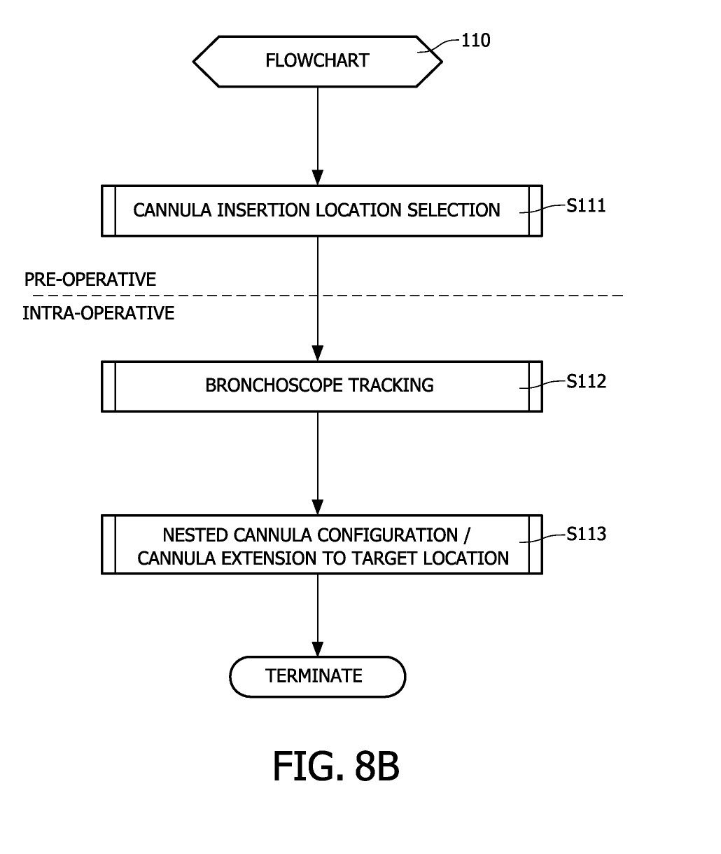

FIG. 7 illustrates an exemplary embodiment of a minimally invasive surgical system derived from the present invention.

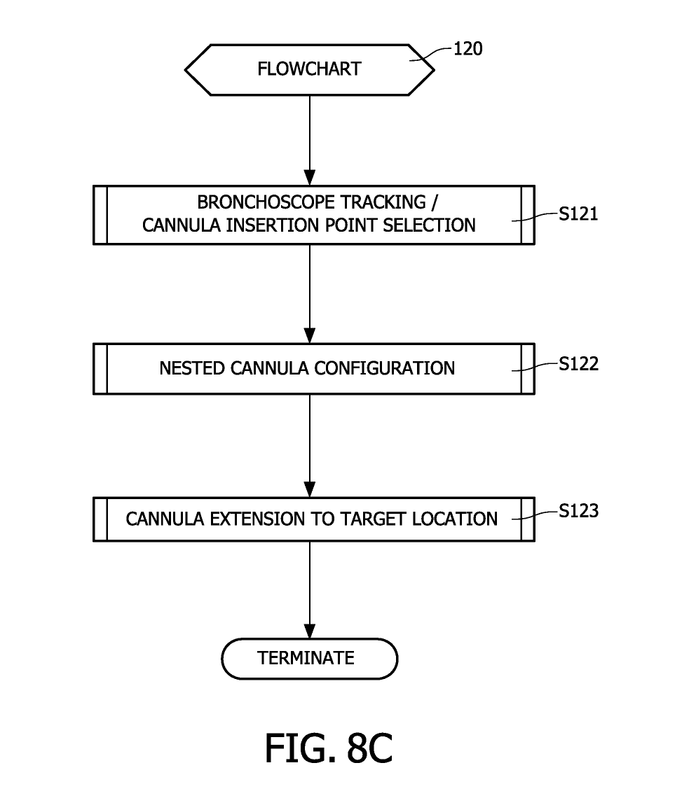

FIGS. 8A-8C illustrate flowcharts representative of exemplary embodiments of the minimally invasive surgical method shown in FIG. 4 derived from the present invention.

As previously stated herein, the present invention is premised on the use of an instrument channel of an endoscope to advance nested cannula tubes towards hard-to-reach lesions within the body. The endoscope is used to explore conventionally reachable areas, whereas the nested cannula performs the final expansion to arrive at the desired target location. One benefit of the present invention is the capability of planning a configuration of the nested cannula and precisely navigating the endoscope/nested cannula through small orifices to small lesions. The present invention may be used with any type of endoscope having an instrument channel (e.g. a bronchoscope, a colonoscope, a laryngoscope, etc), and the nested cannula may be used as a guide for optical fibers (diagnostic), drug delivery or therapy delivery (e.g. guide for micro-instruments, gripping instrument, or phototherapy).

For example, FIG. 3 illustrates bronchoscope 10 and nested cannula 20 for purposes of demonstrating the premise of the present invention. In tandem, the telescoping tubes 21-24 of nested cannula 20 are configured to reach a target location within a bronchial tree 41 of patient 40 via an instrument channel of bronchoscope 10.

More particularly, FIG. 4 illustrates a flowchart 50 representative of a minimally invasive surgical method of the present invention that will now be described herein in the context of bronchoscope 10 and nested cannula 20. A stage S51 of flowchart 50 encompasses a navigation of a distal end 11 of bronchoscope 10 to a cannula insertion location defining a position and orientation of distal end 11 relative to bronchial tree 41. For example, as shown in FIG. 5, distal end 11 of bronchoscope 10 is navigated along a path within bronchial tree 41 to a cannula insertion location 60. The cannula insertion location 60 is either pre-operatively planned or intra-operatively determined as will be further explained herein in connection with the description of FIGS. 8A-8C.

Cannula insertion location 60 defines a position and orientation of distal end 11 within the bronchial tree 41 for facilitating access by nested cannula 20 to a target location 61 within bronchial tree 41 during a stage S52 of flowchart 50. Specifically, stage S52 encompasses an insertion of nested cannula 20 through an instrument channel of bronchoscope 10 with the telescoping tubes of nested cannula 20 being structurally configured to reach target location 61 from cannula insertion location 60. For example, as shown in FIG. 6, nested cannula 20 is extended from an instrument channel 12 of bronchoscope 10. The cannula insertion location 60 (FIG. 5) of distal end 11 of bronchoscope 10 facilitates telescoping tubes 21-24 reaching target location 61 (FIG. 5). The configuration of nested cannula 20 may be pre-operatively planned or intra-operatively determined as will be further explained herein in connection with the description of FIGS. 8A-8C.

In practice, the present invention does not impose any limitation or any restrictions to the implementation of flowchart 50. Thus, the following description of a minimally invasive surgical system of the present invention as shown in FIG. 7 executing three (3) exemplary embodiments of flowchart 50 as shown in FIGS. 8A-8C is provided to facilitate a further understanding of the present invention.

The system of FIG. 7 is described in the context of the previous description of bronchoscope 10 and nested cannula 20 in connection with FIGS. 3-6. Specifically, the system employs an endoscope tracking unit 70 as known in the art for tracking movement of bronchoscope 10 within bronchial tree 41 of patient 40, a nested cannula configuration unit 80 for configuring nested cannula 20 and selecting/determining the cannula insertion location within bronchial tree 41, and an imaging unit 90 as known in the art for displaying intra-operative images of bronchial tree 41 including bronchoscope 10 and nested cannula 20 (e.g., the image of FIG. 5).

FIG. 8A illustrates a flowchart 100 including a pre-operative stage S101 and intra-operative stage S102 and S103. Stage S101 encompasses a selection of a cannula insertion location and a configuration of nested cannula 20 derived from the selection of the cannula insertion location as well as the target location. For example, as shown in FIG. 5, cannula insertion location 60 is selected by a user via an image of tree 41, and an automatic algorithm plans the NC configuration from cannula insertion location 60. In one embodiment, the system and method of International Publication no. WO 2008/032230 A1, Mar. 20, 2008, entitled “Active Cannula Configuration for Minimally Invasive Surgery” to Karen I. Trovato, the entirety of which is incorporated herein by reference, is modified to utilize the cannula insertion location 60 as the starting location for planning an NC configuration. This planning can involve a minimal set of pre-shaped cannula. For example, tube 21 of nested cannula 20 may be a flexible tube that is used to advance nested cannula 20 through instrument channel 12 of bronchoscope 10 and the remaining cannula 22-24 of nested cannula 20 may be pre-shaped arcs tubes and/or straight tubes.

Upon commencing the surgery, stage S102 encompasses a tracking of bronchoscope 20 to cannula insertion location 60. For example, as shown in FIGS. 5 and 7, endoscope tracking unit 70 (e.g., electromagnetic, optical or imaging) is utilized to track bronchoscope 10 as bronchoscope 10 is navigated along within tree 41 from a larynx location to cannula insertion location 60.

Upon reaching cannula insertion location 60, stage S103 encompasses an insertion of nested cannula 20 through an instrument channel 12 of bronchoscope 10 to reach target location 61. For example, as shown in FIGS. 5-7, tube 21 having tubes 22-24 nested therein is advanced through instrument channel 12 of bronchoscope 10 to distal end 11 whereby tubes 21-24 are extended from cannula insertion location 60 to reach target location 61.

FIG. 8B illustrates a flowchart 110 including a pre-operative stage S111 and intra-operative stage S112 and S113. Stages S111-S113 are synonymous with stages S101-S103 (FIG. 8A) except the configuration nested cannula 20 is executed in intra-operative stage S113.

FIG. 8C illustrates a flowchart 120 including intra-operative stages S121-S123. Upon commencing the surgery, stage S121 encompasses a tracking of bronchoscope 10 through bronchial tree 41 whereby a user can select the cannula insertion location 60 while viewing an image of the tracked bronchoscope 10 via imaging unit 90. For example, as shown in FIGS. 5 and 7, endoscope tracking unit 70 (e.g., electromagnetic, optical or imaging) is utilized to track bronchoscope 10 as bronchoscope 10 is navigated from a larynx location in a direction of target location 61 whereby the user ceases navigating bronchoscope 10 upon selecting cannula insertion location 60 which is in suitable proximity of target location 61. Thereafter, stage S122 encompasses a configuration by unit 80 of nested cannula 20 derived from the selected cannula insertion location 60 and target location 61, and stage S123 involves inserting nested cannula 20 through the instrument channel 11 of bronchoscope 10 and extending tubes 21-24 to target location 61.

From FIGS. 3-8C, those having ordinary skill in the art will appreciate many benefits of the present invention including, but not limited to, smaller endoscopes may be used to reach small deep-seeded lesions, the pre-planning of the instrument path facilitates a straightforward approach to reaching the lesions, and no additional hardware is necessary for access to the lesions by nested cannula in view of the cannula insertion location. Furthermore, those having ordinary skill in art will appreciate how to implement the present invention for any type of endoscope as well as various nested cannula configurations.

While various embodiments of the present invention have been illustrated and described, it will be understood by those skilled in the art that the methods and the system as described herein are illustrative, and various changes and modifications may be made and equivalents may be substituted for elements thereof without departing from the true scope of the present invention. In addition, many modifications may be made to adapt the teachings of the present invention to entity path planning without departing from its central scope. Therefore, it is intended that the present invention not be limited to the particular embodiments disclosed as the best mode contemplated for carrying out the present invention, but that the present invention include all embodiments falling within the scope of the appended claims.

1. A method for accessing a target location (61) relative to an anatomical region (41), the method comprising:
navigating a distal end (11) of an endoscope (10) to a cannula insertion location (60) defining a position and an orientation of the distal end (11) of the endoscope (10) relative to the anatomical region (41); and
inserting a nested cannula (20) through an instrument channel (12) of the endoscope (10) to the target location (61), wherein the nested cannula (20) includes a plurality of telescoping tubes (21-24) structurally configured to reach the target location (61) relative to the cannula insertion location (60).

2. The method of claim 1, wherein the navigating of the distal end (11) of the endoscope (10) to the cannula insertion location (60) relative to the target location (61) includes:
pre-operatively selecting the cannula insertion location (60); and
intra-operatively tracking the navigation of the distal end (11) of the endoscope (10) to the cannula insertion location (60).

3. The method of claim 2, wherein the inserting of the nested cannula (20) through the instrument channel (12) of the endoscope (10) to the target location (61) includes:
pre-operatively deriving a configuration of the nested cannula (20) from the cannula insertion location (60) and the target location (61).

4. The method of claim 2, wherein the inserting of the nested cannula (20) through the instrument channel (12) of the endoscope (10) to the target location (61) includes:
intra-operatively deriving a configuration of the nested cannula (20) from the cannula insertion location (60) and the target location (61)

5. The method of claim 1, wherein the navigating of the distal end (11) of the endoscope (10) to the cannula insertion location (60) relative to the target location (61) includes:
intra-operatively tracking the navigation of the distal end (11) of the endoscope (10) in a direction of the target location (61); and
intra-operatively determining the cannula insertion location (60) in proximity of the target location (61).

6. The method of claim 5, wherein the inserting of the nested cannula (20) through the instrument channel (12) of the endoscope (10) to the target location (61) includes:
intra-operatively deriving a configuration of the nested cannula (20) from the cannula insertion location (60) and the target location (61).

7. A device for accessing a target location (61) relative to an anatomical region (41), the system comprising:
an endoscope (10) having a distal end (11) operable to reach an cannula insertion location (60) defining a position and an orientation of the distal end (11) of the endoscope (10) relative to the anatomical region (41); and
a nested cannula (20) operable to be inserted through an instrument channel (12) of the endoscope (10) to the target location (61), wherein the nested cannula (20) includes a plurality of telescoping tubes (21-24) structurally configured to reach the target location (61) relative to the cannula insertion location (60).

8. The device of claim 7, wherein:
the endoscope (10) is a bronchoscope;
the plurality of telescoping tubes (21-24) includes a flexible tube as a largest tube (21) and the remaining tubes (22-24) as pre-shaped tubes.

9. A system for accessing a target location (61) relative to an anatomical region (41), the system comprising:
an endoscope (10) having a distal end (11) operable to reach an cannula insertion location (60) defining a position and an orientation of the distal end (11) of the endoscope (10) relative to the anatomical region (41);
a nested cannula (20) operable to be inserted through an instrument channel (12) of the endoscope (10) to the target location (61), wherein the nested cannula (20) includes a plurality of telescoping tubes (21-24) structurally configured to reach the target location (61) relative to the cannula insertion location (60); and
an imaging unit (90) operable to display intra-operative images of the anatomical region (41) including the endoscope (10) and the nested cannula (20).

10. The system of claim 9, further comprising:
a nested cannula configuration unit (80) operable to pre-operatively select the cannula insertion location (60); and
an endoscope (10) tracking unit (70) operable to intra-operatively track the navigation of the distal end (11) of the endoscope (10) to the cannula insertion location (60).

11. The system of claim 10, wherein the nested cannula configuration unit (80) is further operable to pre-operatively derive a configuration of the nested cannula (20) from the cannula insertion location (60) and the target location (61).

12. The system of claim 10, wherein the nested cannula configuration unit (80) is further operable to intra-operatively deriving a configuration of the nested cannula (20) from the cannula insertion location (60) and the target location (61).

13. The system of claim 9, further comprising:
an endoscope (10) tracking unit (70) operable to intra-operatively track the navigation of the distal end (11) of the endoscope (10) in a direction of the target location (61).

14. The system of claim 13, further comprising:
a nested cannula configuration unit (80) operable to intra-operatively determine the cannula insertion location (60) in proximity of the target location (61).

15. The system of claim 14, wherein the nested cannula configuration unit (80) is further operable to intra-operatively deriving a configuration of the nested cannula (20) from the cannula insertion location (60) and the target location (61).
